(19) United States
(12) Patent Application Publication
(10) Pub. No.: US 2020/0376303 A1
(43) Pub. Date: Dec. 3, 2020

(54) Functional drink and method for preparing the same

(71) Applicant: Fuzhou RenLiang Biological Product Co., Ltd., Fuzhou (CN)

(72) Inventor: Lanqin Yang, Fuzhou (CN)

(73) Assignee: Fuzhou RenLiang Biological Product Co., Ltd.

(21) Appl. No.: 16/558,350

(22) Filed: Sep. 3, 2019

(30) Foreign Application Priority Data
May 29, 2019 (CN) 201910455089.3

(51) Int. Cl. A61P 15/08; A61K 45/06; A61K 36/232; A61K 35/32; A61K 36/804; A61K 36/64

(52) U.S. Cl. CPC A61P 15/08; A61K 45/06; A61K 36/232; A61K 2236/53; A61K 36/804; A61K 36/64; A23V 2002/00; A61K 35/32

(57) ABSTRACT

The present invention discloses the functional drink and method for preparing the same, which is characterized in that: it is made of the main raw materials with the following weight ratios: Deerhorn Glue of 5-8 kg, Rehmannia Glutinosa Libosch of 0.4-0.7 kg, Radix Rehmanniae Preparata of 0.4-0.7 kg, Chinese Angelica of 0.4-0.7 kg, Herba Cistanches of 0.4-0.7 kg, Cuscuta Europaea of 0.4-0.7 kg, Ganoderma Lucidum of 1.8-4 kg, Largehead Atractylodes Rhizome of 0.7 kg, Ligustrum Lucidum of 0.5-0.8 kg, Tortoise Carapace and Plastron of 0.5-0.8 kg, Astragali Radix of 0.6 kg, Fructo-oligosaccharide of 7-9 kg and drinking water of 82.2 L; the “Jun” (emperor) medicinal herbs used in the present invention have the effect of tonifying the kidney and nourishing the essence of blood, and the “Chen” (minister) medicinal herbs have the effect of regulating bodily functions, and the “Zuo” (adjuvant) and “Shi” (courier) medicinal herbs have the effects of adjusting Qi and blood and coordinating various medicines.

### TECHNICAL FIELD

The invention relates to the technical field of medicine, and more particularly relates to the functional drink and method for preparing the same.

### BACKGROUND OF THE INVENTION

With the continuous advancement of the times, people's daily life level has gradually improved, but because of the fast pace of life, people's exercise is very small, often triggering unnecessary diseases, especially in fertility, people often have the problems of low fertility, low pregnancy rate.

Therefore, how to provide the functional drink and method for preparing the same thereof is a problem to be solved by those skilled in the art.

### SUMMARY OF THE INVENTION

In view of this, the present invention provides the functional drink and method for preparing the same.

In order to achieve the above object, the present invention provides the following technical solutions: it is characterized in that: it is made of the main raw materials with the following weight ratios: Deerhorn Glue of 5-8 kg, Rehmannia Glutinosa Libosch of 0.4-0.7 kg, Radix Rehmanniae Preparata of 0.4-0.7 kg, Chinese Angelica of 0.4-0.7 kg, Herba Cistanches of 0.4-0.7 kg, Cuscuta Europaea of 0.4-0.7 kg, Ganoderma Lucidum of 1.8-4 kg, Largehead Atractylodes Rhizome of 0.7 kg, Ligustrum Lucidum of 0.5-0.8 kg, Tortoise Carapace and Plastron of 0.5-0.8 kg, Astragali Radix of 0.6 kg, Fructo-oligosaccharide of 7-9 kg and drinking water of 82.2 L.

Preferably, in the above-mentioned functional drink and method for preparing the same, the steps are as follows:
1) Weighing each raw material;
2) Grinding, extracting and sterilizing the weighed raw materials;
3) Filling and sealing the processed raw materials.

Preferably, in the above-mentioned functional drink and method for preparing the same, the Deerhorn Glue, the Herba Cistanches and the Tortoise Carapace and Plastron are the “Jun” (emperor) medicinal herbs; the Ganoderma Lucidum, the Cuscuta Europaea and the Ligustrum Lucidum are the “Chen” (minister) medicinal herbs; the Rehmannia Glutinosa Libosch, the Radix Rehmanniae Preparata, Largehead Atractylodes Rhizome, Astragali Radix and Chinese Angelica are the “Zuo” (adjuvant) and “Shi” (courier) medicinal herbs.

Preferably, in the above-mentioned functional drink and method for preparing the same, the content of the “ Jun ” (emperor) medicinal components are greater than the content of the “Chen” (minister) medicinal components, and the content of the “Chen” (minister) medicinal components are greater than the content of the “Zuo” (adjuvant) and “Shi” (courier) medicinal components.

According to the above technical solutions, compared with the prior art, the present invention provides the functional drink and method for preparing the same, and the “Jun” (emperor) medicinal herbs used in the present invention have the effect of tonifying the kidney and nourishing the essence of blood, and the “Chen” (minister) medicinal herbs have the effect of regulating bodily functions, and the “Zuo” (adjuvant) and “Shi” (courier) medicinal herbs have the effects of adjusting Qi and blood and coordinating various medicines. Moreover, the raw materials of this invention are natural, and any additives harmful to the human body isn't added, so that this invention has the effect of effectively improving fertility.

### DETAILED DESCRIPTION

The technical solutions in the embodiments of the present invention are clearly and completely described in the following with reference to the accompanying drawings in the embodiments of the present invention. It is obvious that the described embodiments are only a part of the embodiments of the present invention, but not all embodiments. Based on the embodiments of the present invention, all other embodiments obtained by those skilled in the art without creative efforts are within the scope of the present invention.

The invention is characterized in that: it is made of the main raw materials with the following weight ratios: Deerhorn Glue of 5-8 kg, Rehmannia Glutinosa Libosch of 0.4-0.7 kg, Radix Rehmanniae Preparata of 0.4-0.7 kg, Chinese Angelica of 0.4-0.7 kg, Herba Cistanches of 0.4-0.7 kg, Cuscuta Europaea of 0.4-0.7 kg, Ganoderma Lucidum of 1.8-4 kg, Largehead Atractylodes Rhizome of 0.7 kg, Ligustrum Lucidum of 0.5-0.8 kg, Tortoise Carapace and Plastron of 0.5-0.8 kg, Astragali Radix of 0.6 kg, Fructo-oligosaccharide of 7-9 kg and drinking water of 82.2 L.

In order to further optimize the above technical solutions, the steps are as follows:
1) Weighing each raw material;
2) Grinding, extracting and sterilizing the weighed raw materials;
3) Filling and sealing the processed raw materials.

In order to further optimize the above technical solutions, the Deerhorn Glue, the Herba Cistanches and the Tortoise Carapace and Plastron are the “Jun” (emperor) medicinal herbs; the Ganoderma Lucidum, the Cuscuta Europaea and the Ligustrum Lucidum are the “Chen” (minister) medicinal herbs; the Rehmannia Glutinosa Libosch, the Radix Rehmanniae Preparata, Largehead Atractylodes Rhizome, Astragali Radix and Chinese Angelica are the “Zuo” (adjuvant) and “Shi” (courier) medicinal herbs.

In order to further optimize the above technical solutions, the content of the “Jun” (emperor) medicinal components are greater than the content of the “Chen” (minister) medicinal components, and the content of the “Chen” (minister) medicinal components are greater than the content of the “Zuo” (adjuvant) and “ Shi” (courier) medicinal components.

In order to further optimize the above technical solutions, both men and women can take it.

In order to further optimize the above technical solutions, 100-200 ml are taken in the morning, evening and evening.

The various embodiments in the present specification are described in a progressive manner, and each embodiment focuses on differences from other embodiments, and the same or similar parts between the various embodiments may be referred to each other. For the device disclosed in the embodiments, since it corresponds to the method disclosed in the embodiments, the description is relatively simple, and the relevant parts can be referred to the method part.

The above descriptions of the disclosed embodiments enable those skilled in the art to implement or use the invention. Various modifications to these embodiments are obvious to those skilled in the art, and the general principles defined herein may be implemented in other embodiments without departing from the spirit or scope of the invention. Therefore, the present invention is not to be limited to the embodiments shown herein, but applies to the widest extent consistent with the principles and novel features disclosed herein.

1. The functional drink and method for preparing the same, which is characterized in that: Deerhorn Glue of 5-8 kg, Rehmannia Glutinosa Libosch of 0.4-0.7 kg, Radix Rehmanniae Preparata of 0.4-0.7 kg, Chinese Angelica of 0.4-0.7 kg, Herba Cistanches of 0.4-0.7 kg, Cuscuta Europaea of 0.4-0.7 kg, Ganoderma Lucidum of 1.8-4 kg, Largehead Atractylodes Rhizome of 0.7 kg, Ligustrum Lucidum of 0.5-0.8 kg, Tortoise Carapace and Plastron of 0.5-0.8 kg, Astragali Radix of 0.6 kg, Fructo-oligosaccharide of 7-9 kg and drinking water of 82.2 L.

2. The functional drink and method for preparing the same of claim 1, wherein it is characterized in that: the steps are as follows:
1) Weighing each raw material;
2) Grinding, extracting and sterilizing the weighed raw materials;
3) Filling and sealing the processed raw materials.

3. The functional drink and method for preparing the same of claim 1, wherein the Deerhorn Glue, the Herba Cistanches and the Tortoise Carapace and Plastron are the “Jun” (emperor) medicinal herbs; the Ganoderma Lucidum, the Cuscuta Europaea and the Ligustrum Lucidum are the “Chen” (minister) medicinal herbs; the Rehmannia Glutinosa Libosch, the Radix Rehmanniae Preparata, Largehead Atractylodes Rhizome, Astragali Radix and Chinese Angelica are the “Zuo” (adjuvant) and “Shi” (courier) medicinal herbs.

4. The functional drink and method for preparing the same of claim 3, wherein the content of the “Jun” (emperor) medicinal components are greater than the content of the “Chen” (minister) medicinal components, and the content of the “Chen” (minister) medicinal components are greater than the content of the “ Zuo ” (adjuvant) and “ Shi ” (courier) medicinal components.
